(19) United States
(12) Patent Application Publication
(10) Pub. No.: US 2013/0237697 A1
(43) Pub. Date: Sep. 12, 2013

(54) BUILDING BLOCKS AND METHODS FOR THE SYNTHESIS OF 5-HYDROXYMETHYLCYTOSINE-CONTAINING NUCLEIC ACIDS

(71) Applicants: Thomas Carell, Munchen (DE); Martin Munzel, Munchen (DE)

(72) Inventors: Thomas Carell, Munchen (DE); Martin Munzel, Munchen (DE)

(73) Assignee: LUDWIG-MAXIMILIANS-UNIVERSITAT MUNCHEN, Munchen (DE)

(21) Appl. No.: 13/884,007

(22) PCT Filed: Nov. 11, 2011
(86) PCT No.: PCT/EP2011/069954

§ 371 Date: May 8, 2013

(30) Foreign Application Priority Data
Nov. 12, 2010 (EP) 10191078.4

(51) Int. Cl. C07H 19/24; C07H 21/00; C07H 19/06; C07H 1/00

(52) U.S. Cl. CPC C07H 19/24; C07H 1/00; C07H 21/00; C07H 19/06
USPC 536/23.1; 536/27.13; 536/26.7; 536/28.5

(57) ABSTRACT

The present invention relates to building blocks and methods for the efficient synthesis of 5-hydroxymethylcytosine-containing nucleic acids such as DNA or RNA.

### DESCRIPTION

The present invention relates to building blocks and methods for the efficient synthesis of 5-hydroxymethylcytosine-containing nucleic acids such as DNA or RNA.

The genetic material is constructed from the four canonical bases dA, dC, dG, and dT. The dC base is furthermore subject to epigenetic modification. In eucaryotes the dC base is often methylated at position C5 to give the base 5-methylcytosine (^{5-Me}dC).¹ Most recently a new dC-based modification was discovered, which contains instead of the methyl group at position C5 a hydroxymethylene group (FIG. 1).²⁻³ Others and we were able to show that hydroxymethylcytosine is a widespread DNA modification in the brain and that levels vary depending on tissue type.⁴⁻⁵ The function of the new “sixth” base ^{5-HOMe}dC is currently not clear. However, it was shown that particular oxoglutarate dependent TET (ten-to-eleven translocation) oxidases are responsible for its formation.^{3,6} These enzymes specifically oxidize the 5-methyl group of ^{5-Me}dC to give ^{5-HOMe}dC. Recently it was discovered that a lack of these TET enzymes yield misfunctioning stem cells providing a link between formation of the base ^{5-HOMe}dC and cellular development.⁷

In order to facilitate the biochemical investigation of ^{5-HOMe}dC dependent biological processes, an efficient synthesis of ^{5-HOMe}dC containing oligonucleotides (ODNs) is needed. The building block currently used for the synthesis of ^{5-HOMe}dC containing DNA strands necessitates a rather tedious chemical synthesis via an unstable bromo-thymidine intermediate.” In addition, deprotection of the embedded ^{5-HOMe}dC unit requires heating of the synthesized oligonucleotides for 60 h at 60° C. with conc. ammonia,⁹ which prohibits any derivatisation of the oligonucleotides with fluorescence or biotin labels typically needed for many biochemical experiments.

The present application relates to the development of a novel ^{5-HOMe}dC building block, particularly a phosphoramidite building block available in few synthesis steps from a stable and commercially available starting material, e.g. 5-halodeoxycytidine, preferably 5-iododeoxidcytidine. It was found that the building block enables synthesis of ^{5-HOMe}dC-containing nucleic acids using standard phosphoramidite chemistry with excellent coupling yield.

According to the present invention, the amino and hydroxy groups of the ^{5-HOMe}dC base are protected as a cyclic carbamate. This group elegantly inactivates both nucleophilic groups of ^{5-HOMe}dC and is one of the smallest possible protective groups, therefore allowing efficient coupling in the DNA synthesizer. Furthermore it can be easily deprotected simultaneously with cleavage of the DNA strand from the resin by simple base treatment in one step, e.g. by treatment with dilute alkalimetal hydroxide solution under mild conditions, e.g. 12 h at room temperature.

Thus, a first aspect of the present invention relates to a compound having the structural formula (Ia) or (Ib)

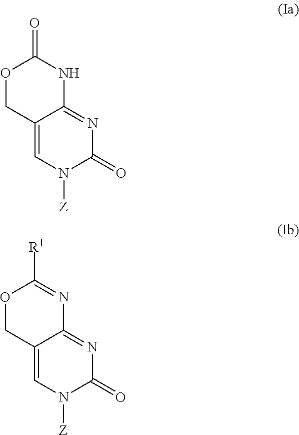

wherein R¹ is a linear or cyclic organic radical having up to 20 carbon atoms, preferably up to 10 carbon atoms, which optionally contains heteroatoms, and Z is H or a cyclic radical.

The compound of formula (Ia) or (Ib) is a protected 5-hydroxymethyl cytosine compound and preferably a protected 5-hydroxymethyl cytidine compound.

In the compound (Ia) or (Ib), Z is preferably a 5- or 6-membered cyclic or heterocyclic radical, particularly a furanosyl or pyranosyl radical, more particularly a ribose, a modified ribose or deoxyribose radical, wherein the 3′-OH group of the ribose, modified ribose or deoxyribose radical may be substituted by a phosphor-containing group, e.g. a phosphate, phosphoester or phosphoramidite group and wherein the 5′-OH group of the ribose, modified ribose analogue or deoxyribose may be substituted by a protection group, e.g. a hydroxy-protection group such as a triphenyl methyl group, preferably a dimethoxytriphenyl methyl group (DMT). The attachment site of the 3′-OH and 5′-OH group can be reversed for use in reverse DNA synthesis.

More preferably, Z is a group having the structural formula (II):

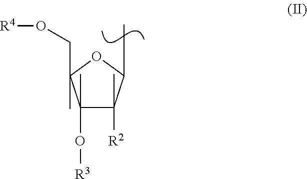

wherein R² is H, OH, halo, azido, CN, —(O)C₁₋₆ (halo) alkyl, —(O)C₂₋₆ (halo) alkenyl, —(O)C₂₋₆ (halo) alkynyl or N(R⁵)₂, wherein R⁵ is in each case independently H, C₁₋₆ (halo) alkyl or phenyl, R³ is H, a hydroxy-protection group, e.g. as indicated above, or a phosphate, phosphoester, phosphoramidite or H-phosphonate group, preferably a phosphoramidite group of formula (III)

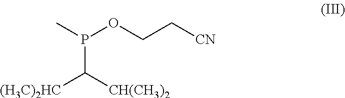

and R⁴ is hydroxy-protection group as indicated above, preferably a triphenylmethyl group such as a dimethoxytriphenylmethyl (DMT) group. The attachment site of R³ and R⁴ can be reversed for use in reverse DNA synthesis.

The group R¹ in formula (Ib) is preferably an aliphatic linear or cyclic group comprising up to 6 C-atoms and optionally up to 2 heteroatoms such as N or O e.g. a linear C₁₋₆ (halo) alkyl group, or a cyclic C₃₋₆ (hetero) alkyl group, or a C₅₋₁₀ aryl or heteroaryl group, e.g. a phenyl or toluyl. optionally substituted by OH, halo, CN, (O)C₁₋₆ (halo) alkyl, a silyl group or N(R⁵)₂, wherein R⁵ is as defined above. Specific examples of R1 are methyl, ethyl, propyl, isopropyl, 2-trifluoroethyl, 2 cyano-ethyl, 2-(trimethyl silyl) ethyl, phenyl or toluyl.

Further, the present invention also refers to formyl or carboxy-protected cytosine or cytidine derivatives which may be used as building blocks or as building block intermediates for the synthesis of 5-hydroxymethylcytosine-containing nucleic acids.

Preferred formyl-protected cytosine or cytidine derivatives have the structural formula (IVa), (IVb) or (IVc),

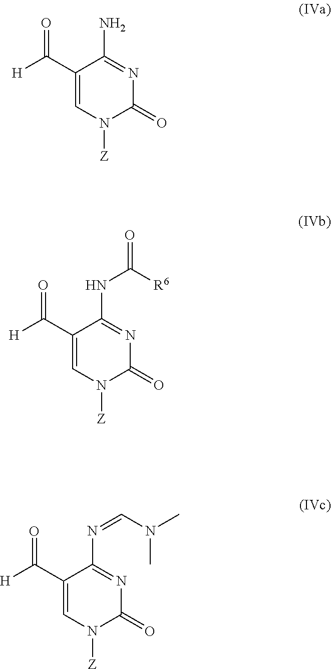

wherein R⁶ is C₁₋₆ (halo) alkyl, e.g. methyl or ethyl, or C₅₋₁₀ aryl or heteroaryl, e.g. phenyl or toluyl, optionally substituted by OH, halo, CN, (O)C₁₋₆ (halo) alkyl or N(R⁵)₂, wherein R⁵ is as defined above, and Z is as defined above (including the preferred embodiments thereof).

Preferred carboxy-protected cytosine or cytidine derivatives have the structural formula (Va), (Vb) or (Vc):

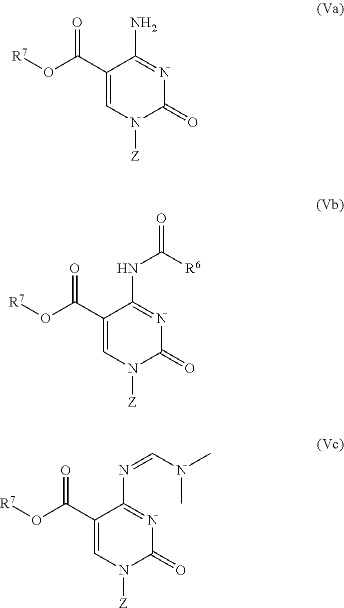

wherein R⁶ is C₁₋₆ (halo) alkyl, e.g. methyl or ethyl, or C₅₋₁₀ aryl or heteroaryl, e.g. phenyl or toluyl, optionally substituted by OH, halo, CN₁(O)C₁₋₆ (halo) alkyl or N(R⁵)₂, wherein R⁵ is as defined above, and Z is as defined above (including the preferred embodiments thereof), and

R⁷ is C₁₋₆ (halo) alkyl or C₅₋₁₀ aryl or heteroaryl, optionally substituted by CN, a silyl group or an aryl such as phenyl, such as methyl, ethyl, propyl, 2-trifluoroethyl, 2-trimethyl silyl-ethyl, phenyl or benzyl, and

Z is as defined above (including the preferred embodiments thereof).

As used herein, the phrase “optionally substituted” means unsubstituted or substituted. The term “substituted” means that a hydrogen atom is removed and replaced by a substituent.

The term “alkyl” refers to straight or branched chain hydrocarbon groups having 1-6, preferably 1-4 carbon atoms. The terms “alkenyl” and “alkynyl” refer to straight or branched chain hydrocarbon groups having 2-6 carbon atoms, preferably 2-4 carbon atoms and at least one CC double or triple bond. Each alkyl, alkenyl or alkynyl group can be substituted with at least one halogen atom.

The terms “halogen” and “halo” refer to fluorine, chlorine, bromine and iodine.

The terms “O alkyl, O alkenyl or O alkynyl” mean alkyl, alkenyl or alkynyl groups bound to an O atom such as methoxy, ethoxy, propoxy, butoxy etc.

The term “cyclic radical” refers to 3-6-membered monocyclic rings or 8-10-membered bicyclic ring systems including fully saturated or unsaturated such as aromatic or non-aromatic cyclic groups which may have at least one heteroatom, e.g. selected from nitrogen atoms, oxygen atoms and/or sulphur atoms.

The terms “furanosyl” and “pyranosyl” refer to 5- or 6-membered cyclic carbohydrate groups.

The term “aryl” refers to phenyl or naphthyl, particularly phenyl. The term “heteroaryl” refers to 5-10-membered heterocyclic systems which include 1-4 heteroatoms selected from N, S and/or O.

A preferred method for the synthesis of the compound (Ia) is depicted in FIG. 2. Starting point is 5-iododeoxycytidine 1,¹⁰ which can be reacted with TBS—Cl to protect the hydroxyl groups. The further synthesis can alternatively be carried out without OH-protection, however the yields of the following reactions are lower and the purification is more tedious. In order to insert the hydroxymethyl group, a Pd-catalyzed formylation reaction with CO is utilized. This reaction is extremely efficient even in the presence of the unprotected exocyclic amino group, providing 2 in yields of above 95%. Next the obtained formyl group at C5 is reduced with NaBH₄ to obtain compound 3. For this step application of Luche conditions is absolutely crucial.¹¹ Without the addition of CeCl₃ the hydride presumably adds to the extremely electrophilic C6 position of the base resulting in decomposition of the starting material 2. To introduce the cyclic carbamate, compound 3 may be treated with 4-nitro-phenolchloroformate¹² to give the protected compound 4 in very good yield. Subsequent deprotection of the silyl groups may be achieved with HF in pyridine. In ethylacetate as solvent, the diol 5 precipitates after completion of the deprotection allowing its isolation by simple centrifugation. Next compound 5 may be converted into the ^{5-HOMe}dC phosphoramidite building block 6 using standard procedures.¹³

Alternative methods for the synthesis of compounds (Ia), (IVa) and (Vb) are shown in FIG. 3.

A further alternative synthesis method for the manufacture of compound (Ia) is shown in FIG. 4.

Compound (Ib) maybe synthesized via condensation with a R¹-trimethoxyacetal (wherein R¹ is as described above) and subsequent transformation to the phosphoramidite as described for (Ia). Compounds (IVb) and (IVc) may be synthesized via Pd-catalyzed formylation and subsequent protection as an amide or DMF acetal. The order of the steps can be reversed. Conversion to the phosphoramidite can be achieved as described for (Ia). Compounds (Va) and (Vc) may be synthesized via Pd catalyzed esterification with R⁷—OH (wherein R⁷ is as described above) and subsequent protection as an amide or DMF acetal. The order of the steps can be reversed. Conversion to the phosphoramidite can be achieved as described for (Ia). These synthesis methods are shown in FIGS. 7A, 7B and 7C.

According to the present invention, it was found that a Pd-catalysed formylation reaction of 5-halodeoxycytidine, preferably 5-iododeoxidcytidine with CO gives 5-formyldeoxycytidine in high yields. Thus, a further aspect of the present invention relates to a method of introducing formyl substituents at position 5 of a cytosine or cytidine compound comprising reacting a 5-halo-substituted starting compound, 5-halocytosine, 5-halocytidine, 5-halodeoxycytidine or protected derivatives thereof with CO under catalysis of Pd.

The building blocks of the present invention can be used for the introduction of 5-hydroxymethylcytosine building blocks in nucleic acids such as DNA or RNA or modified nucleic acids, e.g. sugar and/or phosphate modified nucleic acids. The nucleic acid synthesis may be performed using standard procedures, e.g. standard solid phase chemical synthesis procedures such as the phosphoramidite procedure.

Still a further subject-matter of the present invention is a nucleic acid molecule having incorporated at least one compound as described above, e.g. a compound (Ia), (Ib), (IVa), (IVb), (IVc), (Va), (Vb) and (Vc) as a protected 5-hydroxymethyl cytosine building block. The protection group may be removed under alkaline conditions, preferably in the presence of an alkaline or an alkaline earth metal hydroxide solution, e.g. in a concentration of 0.01-1 mol/l. The alkaline and alkaline earth metals may be selected from Li, Na, K, Rb and Mg. Preferably, Na is used.

Thus, the present invention also refers to a method of removing the cyclic carbamate protective group or alternatively the formyl or carboxylate protecting group, on a compound (Ia), (Ib), (IVa), (IVb), (IVc), (Va), (Vb), (Vc) or a nucleic acid molecule having incorporated at least one compound as indicated comprising a treatment with an aqueous or aqueous/alcoholic alkaline or alkaline earth metal hydroxide solution.

The new ^{5-HOMe}dC building blocks as described above can be incorporated together with alkyne, or norbornene building blocks into DNA and RNA for further click modification, preferably by reaction with a functionalized azide compound which may carry a labelling group. This will allow synthesis of labelled ^{5-HOMe}dC containing oligonucleotides, specifically with biotin of fluorescence labels.

Additionally the formyl-dC building block may in itself allow rapid modification of oligonucleotides by coupling to hydrazine or hydroxylamine containing compounds which may carry a labelling group, e.g. as described above.

Further, the present invention shall be explained in more detail by the following Figures and Examples:

### FIGURES

FIG. 1: Nucleosides present in the mammalian genome

FIG. 2: Synthesis of a cyclic carbamate-protected cytosine phosphoramidite building block 6 and the nucleic acid sequence of ODN1 (C*=^{5HOMe}dc)

FIG. 3: Alternative synthesis methods

FIG. 4: Further alternative synthesis methods

FIG. 5: Nucleosides 7 and 8 were obtained after deprotection of the oligonucleotides using standard NH₃-based conditions. Deprotection with NaOH, however, yields exclusively ^{5-HOMe}dc.

FIG. 6: A) Reversed phase HPLC chromatogram directly after cleavage from the resin (0-50% buffer B in 45 min). B) Reversed phase HPLC chromatogram after cleavage of the DMT group and purification (0-20% buffer B in 45 min). C) MALDI spectrum of the purified strand ODN1. D) Digest of purified DNA strand ODN1.

FIGS. 7A, B and C: Synthesis methods for compounds (Ib), (IVb), (IVc), (Va), (Vc).

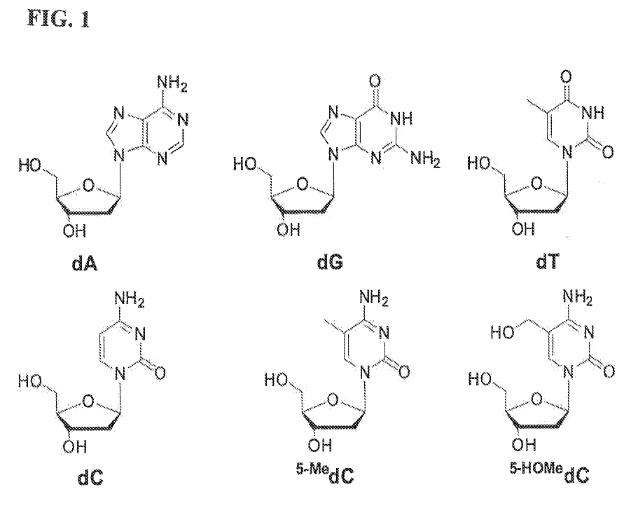

### EXAMPLES

1. General Methods

All non-aqueous reactions were performed using flame- or ovendried glassware under an atmosphere of dry nitrogen. Commercial reagents from Sigma-Aldrich or Acros were used as received unless otherwise noted. Non-aqueous reagents were transferred under nitrogen with a syringe or cannula. Solutions were concentrated in vacuo on a Heidolph rotary evaporator. Chromatographic purification of products was accomplished using flash column chromatography on Merck Geduran Si 60 (40-63 μM) silica gel (normal phase) or Fluka silica gel 100 C₁₈-Reversed phase (15-35 μm). Thin layer chromatography (TLC) was performed on Merck 60 (silica gel F254) plates. Visualization of the developed chromatogram was performed using fluorescence quenching or anisaldehyde staining. ¹H and ¹³C NMR spectra were recorded in deuterated solvents on Bruker ARX 300, Varian VXR400S, Varian Inova 400 and Bruker AMX 600 spectrometers and calibrated to the residual solvent peak. Multiplicities are abbreviated as follows: s=singlet, d=doublet, t=triplet, q=quartet, m=multiplet. ESI spectra and high-resolution ESI spectra were obtained on the mass spectrometers Thermo Finnigan LTQ FT-ICR. Acetonitrile for HPLC-ESI-MS analysis was purchased from VWR, HPLC gradient grade. HCOOH was purchased from Fluka, p.a. for mass spectrometry. MALDI spectra were recorded on a Bruker Autoflex II spectrometer. IR measurements were performed on n Perkin Elmer Spectrum BX FT-IR spectrometer (Perkin Elmer) with a diamond-ATR (Attenuated Total Reflection) setup. Melting points were determined with a Büchi Melting Point B540.

2. Oligonucleotide Synthesis

Oligonucleotide synthesis was performed on an Expedite 8909 Nucleic Acid Synthesis System (PerSeptive Biosystems) using standard DNA synthesis conditions (scale: 1 μM). Phosphoramidites for dA, dC, dG, dT and CPG carriers were obtained from Glen Research. The terminal DMT protecting group was kept on the oligonucleotides after synthesis and removed after cleavage from the resin (see Deprotection and purification). Except for ^{5-HOMe}dC standard coupling conditions were used. For ^{5-HOMe}dC coupling times were doubled to ensure good yields.

3. Deprotection and Purification of Oligonucleotides

Deprotection and cleavage of the oligonucleotides from the CPG carrier was carried out with 0.4 M NaOH solution in MeOH/H₂O 4:1 for 12 h at room temperature. DNA purification was conducted on Waters 2695 analytical HPLC and preparative HPLC Merck Hitachi (L-7150 pump, L-7420 detector) using Nucleosil columns (250*4 mm, C18ec, particle size 3 μm or 250*10 mm, C18ec, 5 μm) from Machery-Nagel. The applied buffer was 0.1 M triethylammoniumacetate in water (buffer A) and 0.1 M triethylammoniumacetate in 80% aqueous MeCN (buffer B). The fractions were checked for purity by analytical HPLC and MALDI-MS. The purified oligonucletides were concentrated using a Christ alpha 2-4 LD plus lyophyllizer. The oligonucleotides still containing the trityl group were deprotected by addition of 100 μL of an 80% acetic acid solution. After incubation at r.t. for 20 min 100 μL of water together with 60 μL of a 3 M solution of sodium acetate were added. The oligonucleotides were purified per preparative HPLC as described above.

4. Enzymatic Digestion

For the enzymatic digestion 1 nmol ODN1 in 100 μL H₂O was mixed with buffer A (10 μL, 300 mM ammonium acetate, 100 mM CaCl₂, 1 mM ZnSO₄, pH 5.7) and nuclease S1 (80 units, aspergillus oryzae) and incubated for 3 h at 37° C. Addition of buffer B (12 μL, 500 mM Tris-HCl, 1 mM EDTA), antarctic phosphatase (10 units), snake venom phosphodiesterase I (0.2 units, Crotalus adamanteus venom) and incubation for further 3 h at 37° C. completed the digestion. The sample was centrifuged (12100 g, 15 min) and analyzed by HPLC (Waters 2695, column: Uptisphere120-3HDO from Interchim). Eluting buffers were buffer A (2 mM NH₄HCOO in H₂O (pH 5.5)) and buffer B (2 mM NH₄HCOO in H₂O/MeCN 20/80). The gradient was 0→12 min; 0%→3% buffer B; 12→60 min; 3%→60% buffer B; 60→62 min; 60%→100% buffer B; 62→70 min; 100% buffer B; 70→85 min; 100→0% buffer B; 85→95 min; 0% buffer B. The elution was monitored at 260 nm.

5. LC-ESI-MS

The samples (100 μL injection volume) were analyzed by LC-ESI-MS on a Thermo Finnigan LTQ Orbitrap XL and were chromatographed by a Dionex Ultimate 3000 HPLC system with a flow of 0.15 mL/min over an Uptisphere120-3HDO column from Interchim. The column temperature was maintained at 30° C. Eluting buffers were buffer C (2 mM HCOONH₄ in H₂O (pH 5.5)) and buffer D (2 mM HCOONH₄ in H₂O/MeCN 20/80 (pH 5.5)). The gradient was 0→12 min; 0%→3% buffer D; 12→60 min; 3%→60% buffer D; 60→62 min; 60%→100% buffer D; 62→70 min; 100% buffer D; 70→85 min; 100→0% buffer D; 85→95 min; 0% buffer D. The elution was monitored at 260 nm (Dionex Ultimate 3000 Diode Array Detector). The chromatographic eluent was directly injected into the ion source without prior splitting. Ions were scanned by use of a positive polarity mode over a full-scan range of m/z 200-1000 with a resolution of 30.000. Parameters of the mass spectrometer were tuned with a freshly mixed solution of adenosine (5 μM) in buffer C. The parameters used in this section were sheath gas flow rate, 16 arb; auxiliary gas flow rate, 11 arb; sweep gas flow rate, 4 arb; spray voltage, 5.0 kV; capillary temperature, 200° C.; capillary voltage, 12 V, tube lens 60 V.

6. Synthetic Procedures for the Phosphoramidite Building Block

### 5-(Iodo)deoxycytidine (1)

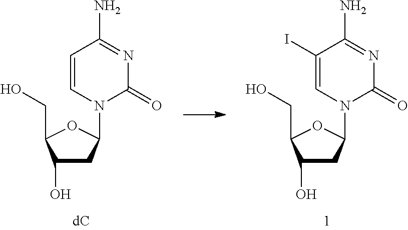 [SMILES: Nc1ccn([C@H]2C[C@@H](O)[C@@H](CO)O2)c(=O)n1 ; Nc1nc(=O)n([C@H]2C[C@@H](O)[C@@H](CO)O2)cc1I]

In a flame dried round bottom flask 10.0 g dC (44.0 mmol, 1.0 eq), 7.70 g iodine (26.4 mmol, 0.6 eq) and 11.4 g mCPBA (70%, 46.2 mmol, 1.05 eq) were dissolved in 120 mL DMF. The reaction mixture was stirred 2 h at room temperature and subsequently evaporated to dryness. (small amounts of DMF are tolerable during subsequent column chromatography) Purification by column chromatography (DCM/MeOH/H₂O/NH₃ 190:10:0.6:0.6→90:10:0.6:0.6) yielded 9.71 g (63%) of 1 as an orange solid.

¹H NMR (400 MHz, CDCl₃/MeOD) δ (ppm)=8.46 (s, 1H), 6.13 (t, ³J=6.0, 1H), 4.34 (dt, ³J=4.7, ³J=6.3, 1H), 3.93 (dt, ³J=3.0, ³J=4.3, 1H), 3.84 (dd, ³J=3.0 Hz, ²J=12.1, 1H), 3.72 (dd, ³J=3.2, ²J=12.1, 1H), 2.39 (ddd, ³J=4.8, ³J=6.3, ²J=13.7, 1H), 2.20-2.09 (m, 1H).

¹³C NMR (101 MHz, MeOD) δ (ppm)=163.9, 153.9, 150.9, 89.5, 88.3, 71.5, 62.2, 56.2, 42.5. HRMS (ESI+) calculated for C₉H₁₃IN₃O₄⁺[M+H]⁺: 353.9945, found: 353.9944. melting range: 133° C.-135° C. (decomposition) IR (ATR): 3191 (w), 1718 (m), 1642 (s), 1286 (m), 1087 (s), 957 (s), 750 (m).

### 3′,5′-(tertbutyldimethylsilyl)-5-(iodo)deoxycytidine (9)

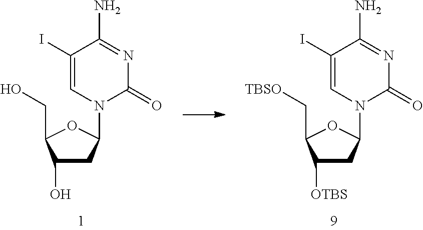 [SMILES: Nc1nc(=O)n([C@H]2C[C@@H](O)[C@@H](CO)O2)cc1I ; CC(C)(C)[Si](C)(C)OC[C@H]1O[C@@H](n2cc(I)c(N)nc2=O)C[C@H]1O[Si](C)(C)C(C)(C)C]

In a flame dried round bottom flask 5.00 g 1 (13.5 mmol, 1.0 eq), 4.16 g imidazole (60.5 mmol, 4.5 eq) and 6.24 g (40.4 mmol, 3.0 eq) TBS-Cl were dissolved in 80 mL DMF and stirred at RT for 16 h. Subsequently the reaction was stopped by the addition of 150 mL sat. NaHCO₃ and extracted with 300 mL CHCl₃. The organic layers were washed with 300 mL H₂O, dried over MgSO₄ and the solvent removed in vacuo. The crude product was purified by column chromatography (DCM/MeOH 99:1→49:1) to yield 6.25 g (80%) of 9 as a slightly yellow solid.

¹H NMR (400 MHz, CDCl₃) δ (ppm)=8.06 (s, 1H), 6.25 - 6.19 (m, 1H), 4.34 (dt, ³J=2.9, ³J=5.9, 1H), 3.97 (q, ³J=2.6, 1H), 3.87 (dd, ³J=2.6, ²J=11.4, 1H), 3.74 (dd, ³J=2.6, ²J=11.4, 1H), 2.44 (ddd, ³J=3.0, ³J=5.9, ²J=13.3, 1H), 2.00-1.90 (m, 1H), 0.92 (s, 9H), 0.87 (s, 9H), 0.13 (s, 3H), 0.12 (s, 3H), 0.06 (s, 3H), 0.05 (s, 3H). ¹³C NMR (101 MHz, CDCl₃) δ (ppm)=163.2, 154.3, 146.7, 88.3, 86.8, 72.2, 62.8, 56.2, 42.6, 26.1, 25.7, 18.5, 18.0, −4.6, −4.9, −5.2, −5.3. HRMS (ESI+): calculated for C₂₁H₄₁IN₃O₄Si₂⁺[M+H]⁺: 582.1675, found: 582.1683. melting range: 196° C.-198° C. IR (ATR): 2929 (w), 2857 (w), 1649 (m), 1470 (m), 1256 (m), 1086 (m), 829 (s), 776 (s).

### 3′,5′-(tertbutyldimethylsilyl)-5-(formyl)deoxycytidine (2)

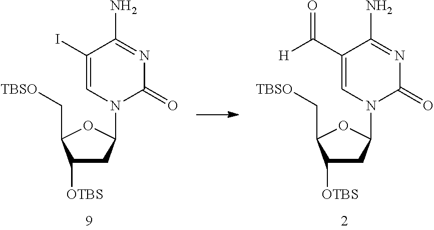 [SMILES: CC(C)(C)[Si](C)(C)OC[C@H]1O[C@@H](n2cc(I)c(N)nc2=O)C[C@H]1O[Si](C)(C)C(C)(C)C ; CC(C)(C)[Si](C)(C)OC[C@H]1O[C@@H](n2cc(C=O)c(N)nc2=O)C[C@H]1O[Si](C)(C)C(C)(C)C]

In a high pressure glass autoclave 3.50 g 9 (6.02 mmol, 1.0 eq), 947 mg PPh₃ (3.61 mmol, 0.6 eq) and 623 mg Pd₂(dba)₃*CHCl₃ (0.60 mmol, 0.1 eq) were dissolved in 90 mL toluene. The autoclave was flushed with CO twice to remove residual air and subsequently the reaction stirred at a CO pressure of 3.5 bar at 60° C. With a syringe pump 2.02 mL Bu₃SnH (7.22 mmol, 1.2 eq) were added through a septum at 0.3 mL per hour. After complete addition the reaction mixture was stirred additional 12 hours at 60° C. Subsequently the CO was discharged and the solvent evaporated in vacuo. The crude product was purified by column chromatography (iHex/EtOAc 4:1→2:1→1:1) to yield 2.84 g (97%) 2 as a yellow solid.

¹H NMR (300 MHz, CDCl₃) δ (ppm)=9.51 (s, 1H), 8.57 (s, 1H), 8.37 (s, 1H), 7.46 (s, 1H), 6.19 (t, ³J=6.1, 1H), 4.40-4.32 (m, 1H), 4.08-4.02 (m, 1H), 3.95 (dd, ³J=2.7, ²J=11.7, 1H), 3.78 (dd, ³J=2.6, ²J=11.6, 1H), 2.59 (ddd, ³J=3.6, ³J=5.8, ²J=10.3, 1H), 2.20-2.08 (m, 1H), 0.89 (s, 9H), 0.88 (s, 9H), 0.10 (s, 3H), 0.08 (s, 6H), 0.07 (s, 3H). ¹³C NMR (75 MHz, CDCl₃) δ (ppm)=187.1, 162.1, 153.1, 152.6, 104.9, 88.8, 87.9, 71.5, 62.6, 42.8, 25.9, 25.7, 18.4, 17.9, −4.5, −4.9, −5.2, −5.4. HRMS (ESI+): calculated for C₂₂H₄₂N₃O₅Si₂⁺[M+H]⁺: 484.2658, found: 484.2654. melting range: 150-152° C. IR (ATR): 3365 (w), 2952 (w), 2929 (w), 2857 (w), 1651 (s), 1245 (m), 1083 (s), 829 (s), 776 (s).

### 3′,5′-(tertbutyldimethylsilyl)-5-(hydroxymethylen)deoxycytidine (3)

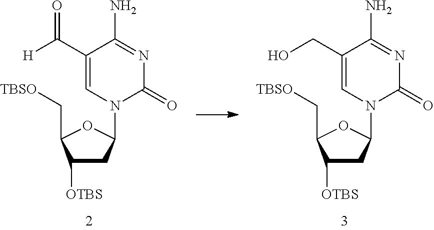 [SMILES: CC(C)(C)[Si](C)(C)OC[C@H]1O[C@@H](n2cc(C=O)c(N)nc2=O)C[C@H]1O[Si](C)(C)C(C)(C)C ; CC(C)(C)[Si](C)(C)OC[C@H]1O[C@@H](n2cc(CO)c(N)nc2=O)C[C@H]1O[Si](C)(C)C(C)(C)C]

In a flame dried round bottom flask 300 mg 2 (0.62 mmol, 1.0 eq) and 707 mg CeCl₃*7 H₂O (1.86 mmol, 3.0 eq) were dissolved in 30 mL methanol. To this solution 24 mg NaBH₄ (0.62 mmol, 1.0 eq) were added and the mixture stirred at room temperature for 30 min. The reaction was stopped by addition of 100 mL sat. NH₄Cl and extracted with 100 mL EtOAc. Subsequently the organic layers were washed twice with 100 mL NH₄Cl, dried over MgSO₄, evaporated to dryness and the crude product purified by column chromatography (DCM/MeOH 19:1, dry loaded) to yield 184 mg 3 (61%) as a colorless oil.

¹H NMR (599 MHz, CDCl₃) δ (ppm)=7.59 (s, 1H), 6.13 (t, ³J=6.4, 1H), 4.39 (d, ²J=13.1, 1H), 4.36 (d, ²J=13.1, 1H), 4.30 (dt, ³J=3.5, ³J=6.6, 1H), 3.90 (q, ³J=3.1, 1H), 3.81 (dd, ³J=3.2, ²J=11.2, 1H), 3.72 (dd, ³J=3.0, ²J=11.3, 1H), 2.36 (ddd, ³J=3.6, ³J=6.1, ²J=13.3, 1H), 1.93 (dt, ³J=6.5, ²J=13.2, 1H), 0.88 (s, 9H), 0.87 (s, 9H), 0.08 (s, 3H), 0.07 (s, 3H), 0.05 (s, 3H), 0.04 (s, 3H). ¹³C NMR (151 MHz, CDCl₃) δ (ppm)=165.2, 156.2, 138.6, 106.0, 87.8, 86.2, 71.7, 62.7, 59.5, 42.2, 25.9, 25.8, 18.4, 18.0, −4.6, −4.9, −5.3, −5.4. HRMS (ESI+): calculated for C₂₂H₄₄N₃O₅Si₂⁺[M+H]⁺: 486.2814, found: 484.2815. IR (ATR): 3193 (m), 3060 (m), 2950 (m), 2928 (m), 2857 (m), 1663 (s) 1485 (s), 1378 (m), 1291 (s), 1100 (s), 829 (s), 776 (s).

### 3′,5′-(tertbutyldimethylsilyl)-4,5-(1,3-[3H,6H]oxazin-2-one)deoxycytidine (4)

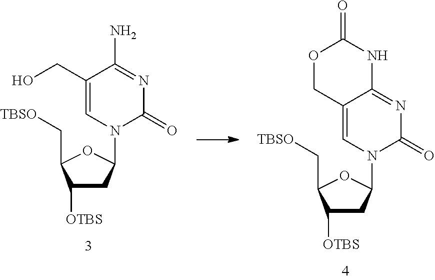 [SMILES: CC(C)(C)[Si](C)(C)OC[C@H]1O[C@@H](n2cc(CO)c(N)nc2=O)C[C@H]1O[Si](C)(C)C(C)(C)C ; CC(C)(C)[Si](C)(C)OC[C@H]1O[C@@H](n2cc3c(nc2=O)NC(=O)OC3)C[C@H]1O[Si](C)(C)C(C)(C)C]

In a flame dried round bottom flask 12 mg (0.02 mmol, 1.0 eq) 3 were dissolved in 5 mL THF and subsequently 5 mg (0.02 mmol, 1.0 eq) 4-Nitrophenylchloroformiate added. The mixture was stirred at room temperature for 90 min. 9 μL (0.05 mmol, 2.0 eq) DIPEA were added and the solution stirred additional 90 min. Afterwards the reaction mixture was evaporated to dryness and the crude product purified by column chromatography (DCM/MeOH 99:1) to yield 11 mg (87%) of 4 a colorless solid.

¹H NMR (599 MHz, CDCl₃) δ (ppm)=8.17 (s, 1H), 6.22 (t, ³J=6.0, 1H), 5.12 (d, ²J=13.2, 1H), 5.09 (d, ²J=13.3, 1H), 4.34 (dd, ³J=4.1, ³J=9.8, 1H), 4.04-3.98 (m, 1H), 3.93 (dd, ³J=2.4, ²J=11.6, 1H), 3.77 (dd, ³J=2.2, ²J=11.5, 1H), 2.57 (ddd, ³J=4.6, ³J=6.0, ²J=13.4, 1H), 2.10-2.00 (m, 1H), 0.91 (s, 9H), 0.88 (s, 9H), 0.11 (s, 3H), 0.10 (s, 3H), 0.07 (s, 3H), 0.06 (s, 3H). ¹³C NMR (75 MHz, CDCl₃) δ (ppm)=159.7, 154.4, 149.8, 138.4, 96.2, 88.2, 87.4, 71.1, 64.6, 62.3, 42.4, 25.8, 25.7, 18.3, 17.9, −4.6, −5.0, −5.42, −5.44. HRMS (ESI+): calculated for C₂₃H₄₂N₃O₆Si₂⁺[M+H]⁺: 512.2607, found: 512.2611. Melting range: 96° C.-97° C. IR (ATR): 2929 (w), 2857 (w), 1758 (m), 1667 (m), 1562 (m), 1251 (m), 1066 (m), 829 (s), 776 (s).

### 4,5-(1,3-[3H,6H]oxazin-2-one)deoxycytidine (5)

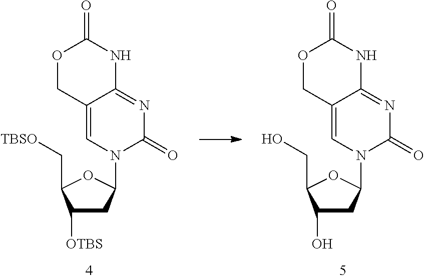 [SMILES: CC(C)(C)[Si](C)(C)OC[C@H]1O[C@@H](n2cc3c(nc2=O)NC(=O)OC3)C[C@H]1O[Si](C)(C)C(C)(C)C ; O=C1Nc2nc(=O)n([C@H]3C[C@@H](O)[C@@H](CO)O3)cc2CO1]

In a polypropylene tube 187 mg 4 (0.37 mmol, 1.0 eq) were dissolved in 25 mL EtOAc, subsequently 147 μL pyridine (1.83 mmol, 5.0 eq) and 157 μL HF*pyridine (70% HF, 5.48 mml, 15.0 eq) were added and the reaction mixture stirred 14 h at room temperature. During this time a white solid precipitated. 500 μL TMSOMe were added and the reaction mixture stirred another 30 min. Subsequently the solid was collected by centrifugation (6000 rpm, 15 min). The supernatant was evaporated to dryness and again treated as described above. The reaction yielded 88 mg (85%) 5 as a colorless solid.

¹H NMR (400 MHz, CD₃OD) δ (ppm)=8.39 (t, ⁵J=1.1, 1H), 6.20 (t, ³J=6.2, 1H), 5.21 (dd, ⁵J=0.9, ²J=13.2, 1H), 5.18 (dd, ⁵J=0.9, ²J=13.2, 1H), 4.37 (dt, ³J=3.9, ³J=6.3, 1H), 4.00 (dd, ³J=3.7, ³J=7.2, 1H), 3.84 (dd, ³J=3.2, ²J=12.2, 1H), 3.75 (dd, ³J=3.8, ²J=12.2, 1H), 2.49 (ddd, ³J=4.2, ³J=6.2, ²J=13.7, 1H), 2.17 (dt, ³J=6.3, ²J=13.7, 1H). ¹³C NMR (101 MHz, CD₃OD) δ (ppm)=162.0, 157.8, 153.0, 140.4, 99.4, 89.6, 88.9, 71.7, 66.2, 62.5, 42.6. HRMS (ESI+): calculated for C₁₁H₁₄N₃O₆⁺[M+H]⁺: 284.0877, found: 284.0877. Melting range: >200° C. decomposition. IR (ATR): 3320 (m), 1745 (m), 1668 (s), 1626 (s), 1499 (s), 1276 (s), 1103 (s) 872 (s).

### 5′-(dimethoxytrityl)-4,5-(1,3-[3H,6H]oxazin-2-one)deoxycytidine (10)

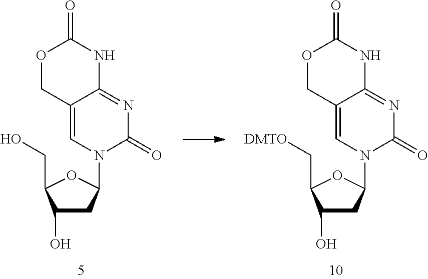

In a flame dried round bottom flask 85 mg (0.30 mmol, 1.0 eq) 5 and 105 mg DMT-Cl (0.30 mmol, 1.0 eq) were dissolved in 10 mL pyridine. The reaction mixture was stirred for 17 h at room temperature and subsequently evaporated to dryness. The crude product was purified by column chromatography (DCM/MeOH 99:1→49:1; 0.1% NEt₃) to yield 75 mg (43%) 10 as a colorless oil.

¹H NMR (300 MHz, CDCl₃) δ (ppm)=8.32 (s, 1H), 7.33-7.14 (m, 9H), 6.77 (d, ³J=8.9, 4H), 6.24 (t, ³J=5.8, 1H), 4.64 (m, 1H), 4.14 (dd, ³J=2.8, ³J=6.6, 1H), 4.08-4.01 (m, 1H), 3.72 (s, 6H), 3.43 (dd, ³J=2.8, ²J=10.8, 1H), 3.37 (dd, ³J=2.8, ²J=10.7, 1H), 2.76-2.65 (m, 2H), 2.35-2.23 (m, 2H).¹³C NMR (75 MHz, CDCl₃) δ (ppm)=159.5, 158.64, 158.60, 158.4, 154.9, 149.9, 144.1, 139.0, 135.0, 134.9, 130.0, 129.9, 128.0, 127.9, 127.1, 113.2, 96.7, 87.1, 86.8, 86.3, 70.3, 63.8, 62.6, 55.13, 55.05, 42.1. HRMS (ESI+): calculated for C₃₂H₃₀N₃O₈⁻[M−H]⁻: 584.2038, found: 584.2033.

### 3′-(diisopropylcyanoethylphospino)-5′-(dimethoxytrityl)-4,5-(1,3-[3H,6H]oxazin-2-one)deoxycytidine (6)

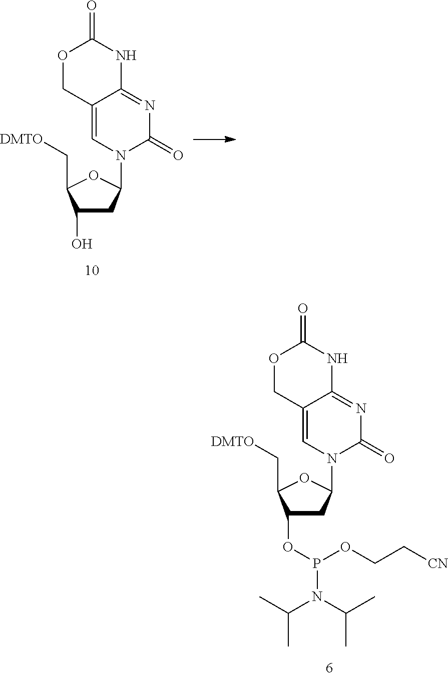

In a flame dried round bottom flask 86 mg (0.15 mmol, 1.0 eq) 10, 13 mg (0.07 mmol, 0.5 eq) diisopropylammoniumtetrazolide and 57 μL (0.18 mmol, 1.2 eq) 2-cyanoethoxy-N,N,N′,N′-tetraisopropylphosphordiamidite were dissolved in rigorously degassed DCM (freeze, pump, thaw). The solution was allowed to stir for 15h at room temperature and was subsequently concentrated to dryness in an argon atmosphere. The crude product was purified by column chromatography (DCM/MeOH 49:1, 0.1% NEt₃). Pure fractions were evaporated to dryness in an argon atmosphere to yield 58 mg (50%) of 6 as a colorless foam.

The compound was air sensitive and was directly used for solid phase DNA synthesis. Its identity was unequivocally proven by successful incorporation into DNA.

7. Oligonucleotide Synthesis

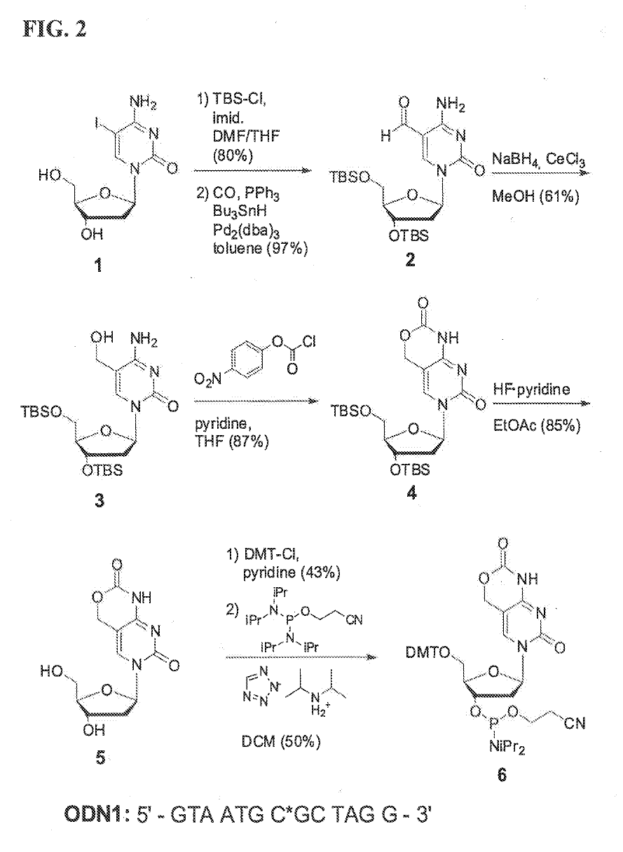

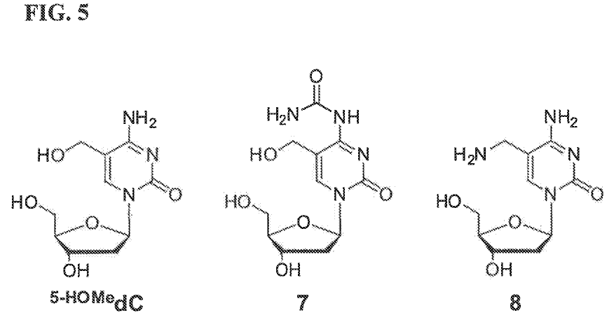

The oligonucleotide ODN1 (FIG. 2) was prepared using the phosphoramidite 6 (C*). Coupling times with 6 were doubled to allow efficient incorporation into the oligonucleotide chain. Initial attempts to deprotect the strands with a standard protocol (conc. ammonia at room temperature overnight) furnished oligonucleotides containing ^{5-HOMe}dC. However, the urea derivative 7 and the aminomethyl-dC nucleobase 8 were formed as major byproducts (FIG. 5). To prevent these undesired side reactions 0.4 M NaOH in MeOH/H₂O 4:1 as the deprotection solution at room temperature overnight. Cleavage of the DNA strand from the solid support and deprotection of all bases including the cyclic carbamate achieved under these conditions, yielding a DNA strand that exclusively contained ^{5-HOMe}dC. Interestingly, related urea derivatives were proven to be stable during DNA synthesis and deprotection.¹⁴

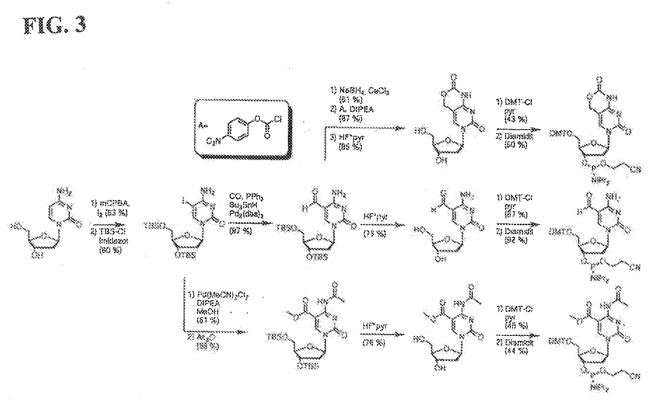

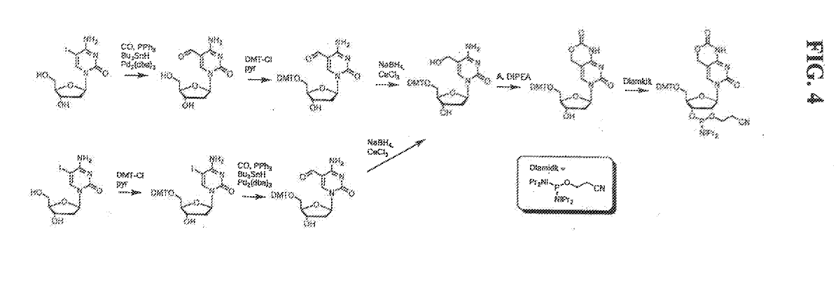

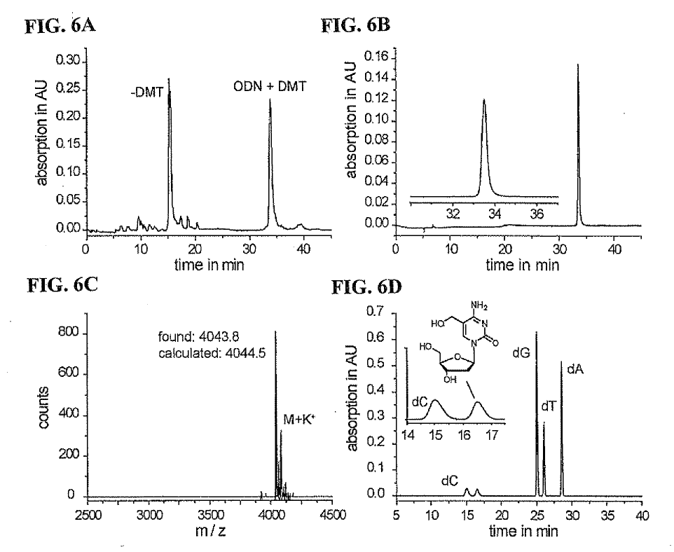

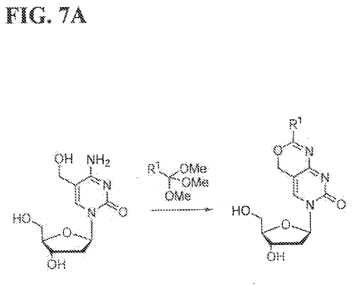

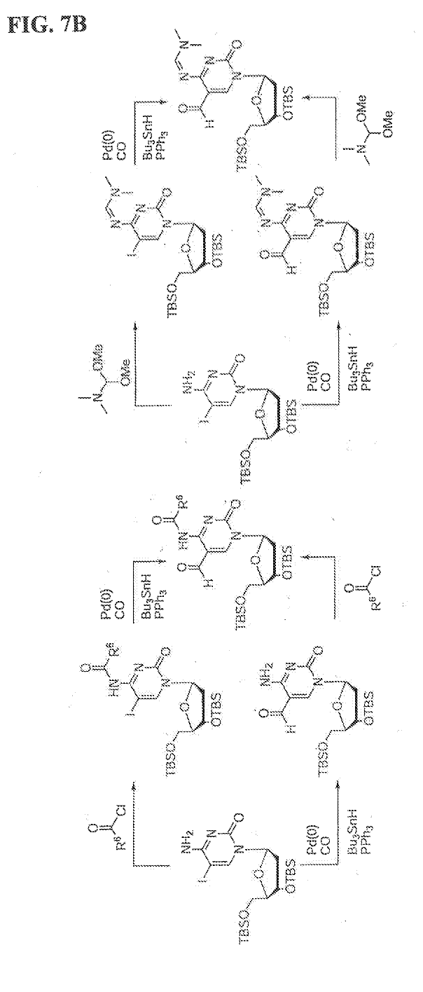

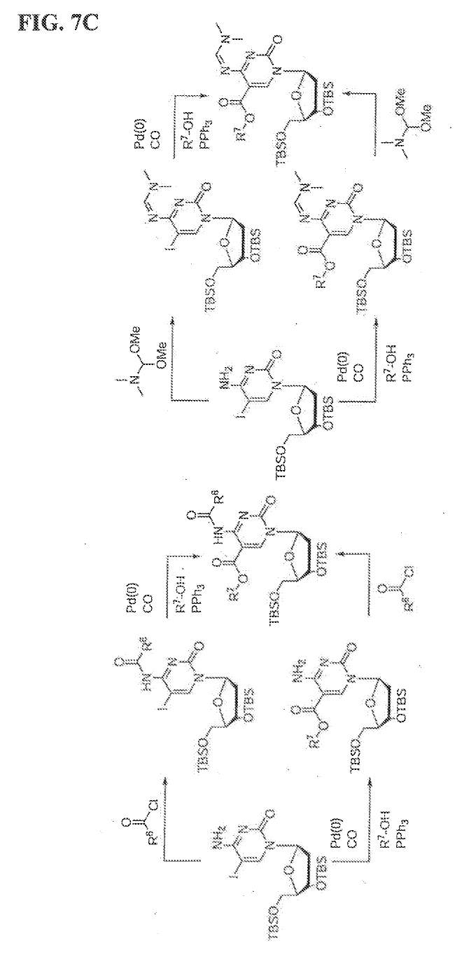

FIG. 6A depicts the raw HPLC chromatogram obtained directly after DNA cleavage and deprotection. The spectrum shows that the building block 6 indeed couples with high efficiency during DNA assembly in the synthesizer. FIG. 3B shows the reversed phase HPLC chromatogram of the purified ^{5-HOMe}dC containing oligonucleotide together with the MALDI-TOF mass spectrum (FIG. 6C) proving the correct incorporation of ^{5-HOMe}dC into the DNA strand. This is noteworthy, because we observed S_{N}2-type reactions at the pseudo-benzylic position of ^{5-HOMe}dC especially under acidic conditions or when the oxygen atom was derivatized with an electron withdrawing group. The unusually high reactivity of the primary OH group initially hampered our attempts to protect ^{5-HOMe}dC as a bis-acetate. This reactivity also explains the formation of the byproduct 8 in the reaction with NH₄OH.

To gain further evidence for the exclusive formation of ^{5-HOMe}dC we conducted an enzymatic digestion study. To this end we treated the obtained oligonucleotide ODN1 first with nuclease S1 for 3 h at 37° C. followed by incubation with antarctic phosphatase and snake venom phosphordiesterase for additional 3 h at 37° C. The obtained digest was analyzed by HPLC-ESI-MS. The chromatogram is depicted in FIG. 6D and shows besides the four canonical bases dA, dC, dG, and dT an additional signal, which features the correct molecular weight for ^{5-HOMe}dC. The high resolution MS data support the molecular formula C₁₀H₁₅N₃O₅ expected for the target compound.

8. Summary

We report a short and efficient synthesis of a novel ^{5-HOMe}dC phosphoramidite building block. The key step in the synthesis is a Pd(O) catalyzed formylation and the simultaneous protection of the primary hydroxyl group together with the exocyclic amino group at the heterocycle as a cyclic carbamate. Deprotection of this unit is conveniently achieved with NaOH solution at mild conditions now enabling the synthesis of ^{5-HOMe}dC oligonucleotides containing additional modifications such as fluorophores or biotin labels. For these purposes the here reported chemistry in combination with the new ability to perform Cu(I) catalyzed click modification or Cu-free modification of DNA and RNA¹⁵⁻¹⁶ should be particularly applicable.

### LITERATURE

¹Law, J. A.; Jacobsen, S. E. Nat. Rev. Genet. 2010, 11, 204-220.
²Kriaucionis, S.; Heintz, N. Science 2009, 324, 929-930.
³Tahiliani, M.; Koh, K. P.; Shen, Y. H.; Pastor, W. A.; Bandukwala, H.; Brudno, Y.; Agarwal, S.; Iyer, L. M.; Liu, D. R.; Aravind, L.; Rao, A. Science 2009, 324, 930-935.
⁴Münzel, M.; Globisch, D.; Bruckl, T.; Wagner, M.; Welzmiller, V.; Michalakis, S.; Müller, M.; Biel, M.; Carell, T. Angew. Chem. Int. Ed. 2010, 49, 5375-5377.
⁵Szwagierczak, A.; Bultmann, S.; Schmidt, C. S.; Spada, F.; Leonhardt, H. Nucleic Acids Res. 2010, 38, e181.
⁶Loenarz, C.; Schofield, C. J. Chem. Biol. 2009, 16, 580-583.
⁷Ito, S.; D'Alessio, A. C.; Taranova, O. V.; Hong, K.; Sowers, L. C.; Zhang, Y. Nature 2010, 466, 1129-1133.
⁸Shiau, G. T.; Schinazi, R. F.; Chen, M. S.; Prusoff, W. H. J. Med. Chem. 1980, 23, 127-133.
⁹Tardy-Planechaud, S.; Fujimoto, J.; Lin, S. S.; Sowers, L. C. Nucleic Acids Res. 1997, 25, 553-558.
¹⁰Hwang, C. H.; Park, J. S.; Won, J. H.; Kim, J. N.; Ryu, E. K. Arch. Pharm. Res. 1992, 15, 69-72.
¹¹Luche, J. L. J. Am. Chem. Soc. 1978, 100, 2226-2227.
¹²Sammet, B.; Synlett 2009, 3050-3051.
¹³Caruthers, M. H. Acc. Chem. Res. 1991, 24, 278-284.
¹⁴Miyata, K.; Tamamushi, R.; Ohkubo, A.; Taguchi, H.; Seio, K.; Santa, T.; Sekine, M. Org. Lett. 2006, 8, 1545-1548.
¹⁵Gierlich, J.; Burley, G. A.; Gramlich, P. M. E.; Hammond, D. M.; Carell, T. Org. Lett. 2006, 8, 3639-3642.
¹⁶Gramlich, P. M. E.; Wirges, C. T.; Manetto, A.; Carell, T. Angew. Chem. Int. Ed. 2008, 47, 8350-8358.

1. A compound having the structural formula (Ia) or (Ib) 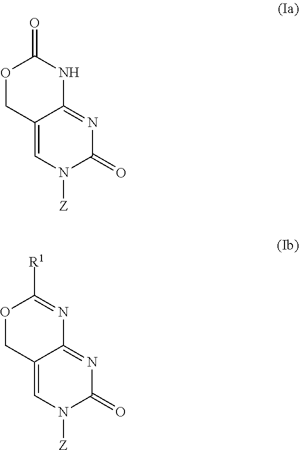
wherein R¹ is a linear or cyclic organic radical having up to 20 carbon atoms which optionally contains heteroatoms, and
Z is H or a cyclic radical.

2. The compound of claim 1, wherein Z is a 5- or 6-membered cyclic radical, particularly a ribose, ribose analogue or deoxyribose radical, wherein the 3′-OH group of the ribose, ribose analogue or deoxyribose radical may be substituted by a phosphor-containing group, e.g. a phosphate, phosphoester or phosphoramidite group and wherein the 5′-OH group of the ribose, ribose analogue or deoxyribose radical may be substituted by a protection group.

3. The compound of claim 1, wherein Z is a group having the structural formula (II): 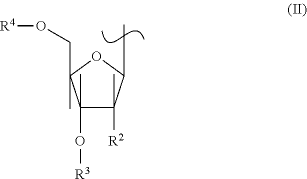
wherein R² is H, OH, halo, azido, CN, —(O)C₁₋₆ (halo) alkyl, —(O)C₂₋₆ (halo) alkenyl, —(O)C₂₋₆ (halo) alkynyl or N(R⁵)₂,
R⁵ is in each case independently H, C₁₋₆ (halo) alkyl or phenyl, and
R³ is H, a hydroxy-protection group or a phosphate, phosphoester, phosphoramidite or H-phosphonate group, preferably a phosphoramidite group of formula (III) 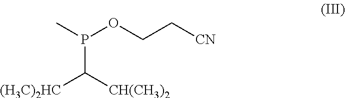
and R⁴ is hydroxy-protection group, preferably a triphenylmethyl protection group such as a dimethoxytriphenylmethyl (DMT) group.

4. The compound of claim 3, wherein R¹ is an aliphatic linear or cyclic group comprising up to 6 C-atoms and optionally up to 2 heteroatoms such as N or O, e.g. a C₁₋₆ (halo) alkyl group, or a C₃₋₆ (hetero) alkyl group, or a C₅₋₁₀ aryl or heteroaryl group optionally substituted by OH, halo, CN, (O)C₁₋₆ (halo) alkyl or N(R⁵)₂, wherein R⁵ is as defined for the compound of formula (II).

5. A method of introducing a formyl substituent at position 5 of a cytosine cytidine, or deoxycytidine compound comprising reacting a 5-halo substituted starting compound with CO under catalysis of Pd.

6. A method for the synthesis of a nucleic acid, comprising incorporating a compound of claim 1 into said nucleic acid.

7. A method of claim 6, wherein the nucleic acid synthesis is carried out by a phosphoramidite procedure.

8. A nucleic acid molecule having incorporated at least one compound of claim 1.

9. A method of removing the cyclic carbamate protective group on a compound having the structural formula (Ia) or (Ib) 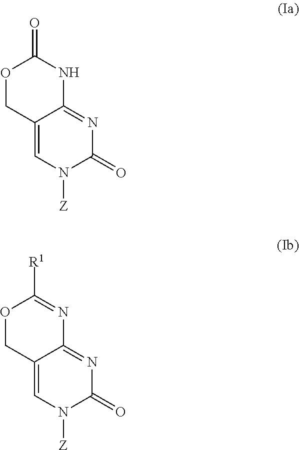
wherein R¹ is a linear or cyclic organic radical having up to 20 carbon atoms which optionally contains heteroatoms, and Z is H or a cyclic radical or a nucleic acid molecule of having incorporated at least one compound of formula (Ia) or (Ib), comprising a treatment with an aqueous or aqueous/alcoholic alkaline or alkaline earth metal hydroxide solution.
